SEQUENCE LISTING (89 sequences)

SEQ ID NO: 1 - DNA, 22 bp, synthetic construct
  source 1..22: mol_type = other DNA
ggcaaactgcccgggcagtcgc

SEQ ID NO: 2 - DNA, 22 bp, synthetic construct
  source 1..22: mol_type = other DNA
gttgttgatggtgcgggccagc

SEQ ID NO: 3 - DNA, 22 bp, synthetic construct
  source 1..22: mol_type = other DNA
gttgttgatggtgtgggccagc

SEQ ID NO: 4 - DNA, 23 bp, synthetic construct
  source 1..23: mol_type = other DNA
cggcggactaccatcacgtcgtg

SEQ ID NO: 5 - DNA, 22 bp, synthetic construct
  source 1..22: mol_type = other DNA
tggaagttcgtgccgggagtac

SEQ ID NO: 6 - DNA, 20 bp, synthetic construct
  source 1..20: mol_type = other DNA
ggcaaactgcccgggcagtc

SEQ ID NO: 7 - DNA, 20 bp, synthetic construct
  source 1..20: mol_type = other DNA
gcgcgggccagcgcggcgcc

SEQ ID NO: 8 - DNA, 22 bp, synthetic construct
  source 1..22: mol_type = other DNA
gtcgctgatggcgcgggccagc

SEQ ID NO: 9 - DNA, 21 bp, synthetic construct
  source 1..21: mol_type = other DNA
tgcgagggtggctcgctggcg

SEQ ID NO: 10 - DNA, 21 bp, synthetic construct
  source 1..21: mol_type = other DNA
tggtcacccgggtcgatgtgg

SEQ ID NO: 11 - DNA, 23 bp, synthetic construct
  source 1..23: mol_type = other DNA
cggcggactaccatcacgtcgtg

SEQ ID NO: 12 - DNA, 20 bp, synthetic construct
  source 1..20: mol_type = other DNA
tggaagtccgcgccgggagt

SEQ ID NO: 13 - DNA, 20 bp, synthetic construct
  source 1..20: mol_type = other DNA
tggaagttcgtgccgggagt

SEQ ID NO: 14 - DNA, 21 bp, synthetic construct
  source 1..21: mol_type = other DNA
gccaccgcattgcgtcagcgg

SEQ ID NO: 15 - DNA, 21 bp, synthetic construct
  source 1..21: mol_type = other DNA
ttctcgtcgactccttcgaag

SEQ ID NO: 16 - DNA, 21 bp, synthetic construct
  source 1..21: mol_type = other DNA
cgtcggcaatcgaggcggtgt

SEQ ID NO: 17 - DNA, 21 bp, synthetic construct
  source 1..21: mol_type = other DNA
acatcgacctcatcgacgccg

SEQ ID NO: 18 - DNA, 21 bp, synthetic construct
  source 1..21: mol_type = other DNA
atatcgacctcatcgacgccg

SEQ ID NO: 19 - DNA, 21 bp, synthetic construct
  source 1..21: mol_type = other DNA
cttcgaaggagtcgacgagaa

SEQ ID NO: 20 - DNA, 21 bp, synthetic construct
  source 1..21: mol_type = other DNA
gtcctcggccgtctggcgcac

SEQ ID NO: 21 - DNA, 22 bp, synthetic construct
  source 1..22: mol_type = other DNA
gcggcaacgcggcgtcgatgag

SEQ ID NO: 22 - DNA, 21 bp, synthetic construct
  source 1..21: mol_type = other DNA
cacacccgctgtcaggtccac

SEQ ID NO: 23 - DNA, 22 bp, synthetic construct
  source 1..22: mol_type = other DNA
atggcttggccaccagggtgct

SEQ ID NO: 24 - DNA, 21 bp, synthetic construct
  source 1..21: mol_type = other DNA
cggttcggcggtgccatcagg

SEQ ID NO: 25 - DNA, 21 bp, synthetic construct
  source 1..21: mol_type = other DNA
tggtttggtggtgtcatcagg

SEQ ID NO: 26 - DNA, 45 bp, synthetic construct
  source 1..45: mol_type = other DNA
ccgcgcgaacgtatggtggacgtatgcgggcgttgatcagcgcgg

SEQ ID NO: 27 - DNA, 44 bp, synthetic construct
  source 1..44: mol_type = other DNA
cgccgctcgtcgacgtgcagaacgacttctgcgagggtgcggcg

SEQ ID NO: 28 - DNA, 35 bp, synthetic construct
  source 1..35: mol_type = other DNA
gcgagggtggctcgctggcggtaaccggtggcgcc

SEQ ID NO: 29 - DNA, 45 bp, synthetic construct
  source 1..45: mol_type = other DNA
ccggcaggtaaccggtggcgccgcgctggcccgctcaactgccgg

SEQ ID NO: 30 - DNA, 36 bp, synthetic construct
  source 1..36: mol_type = other DNA
ccccgcgccatcagcgactacctgaccgaagcgggg

SEQ ID NO: 31 - DNA, 45 bp, synthetic construct
  source 1..45: mol_type = other DNA
ccgcgtcggactaccatcacgtcgtggcaaccaaggactacgcgg

SEQ ID NO: 32 - DNA, 37 bp, synthetic construct
  source 1..37: mol_type = other DNA
cacccggcaaccaaggacttccacatcgacccgggtg

SEQ ID NO: 33 - DNA, 43 bp, synthetic construct
  source 1..43: mol_type = other DNA
tcgcgacccgggtgaccacttctccggcacaccggactcgcgg

SEQ ID NO: 34 - DNA, 45 bp, synthetic construct
  source 1..45: mol_type = other DNA
cgcgcgtattcctcgtcgtggccaccgcattgcgtcagccgcgcg

SEQ ID NO: 35 - DNA, 44 bp, synthetic construct
  source 1..44: mol_type = other DNA
cctgagcgtactcccggcgcggacttccatcccagtcgctcagg

SEQ ID NO: 36 - DNA, 46 bp, synthetic construct
  source 1..46: mol_type = other DNA
cgcgcccccagtctggacacgtcggcaatcgaggcggtgtggcgcg

SEQ ID NO: 37 - DNA, 44 bp, synthetic construct
  source 1..44: mol_type = other DNA
cgcggcgtgttctacaagggtgcctacaccggagcgtagccgcg

SEQ ID NO: 38 - DNA, 43 bp, synthetic construct
  source 1..43: mol_type = other DNA
cggcgctacaagggtgcctacaccggagcgtacagcggcgccg

SEQ ID NO: 39 - DNA, 38 bp, synthetic construct
  source 1..38: mol_type = other DNA
ccgggcttcgaaggagtcgacgagaacggcacgcccgg

SEQ ID NO: 40 - DNA, 38 bp, synthetic construct
  source 1..38: mol_type = other DNA
ccggacgccactgctgaattggctgcggcaacgtccgg

SEQ ID NO: 41 - DNA, 42 bp, synthetic construct
  source 1..42: mol_type = other DNA
cggccccgaccacatcgacctcatcgacgccgcgttgggccg

SEQ ID NO: 42 - DNA, 45 bp, synthetic construct
  source 1..45: mol_type = other DNA
gccgctggtggcaataccgatcacatcgacctcatcgacagcggc

SEQ ID NO: 43 - DNA, 45 bp, synthetic construct
  source 1..45: mol_type = other DNA
cgcgcgccaccgatcattgtgtgcgccagacggccgaggagcgcg

SEQ ID NO: 44 - DNA, 46 bp, synthetic construct
  source 1..46: mol_type = other DNA
cgcggacgcggttcgcaatggcttggccaccagggtgctgtccgcg

SEQ ID NO: 45 - DNA, 41 bp, synthetic construct
  source 1..41: mol_type = other DNA
ccgcgctgctggtggacctgacagcgggtgtgtcggcgcgg

SEQ ID NO: 46 - DNA, 44 bp, synthetic construct
  source 1..44: mol_type = other DNA
cgggcgccgataccaccgtcgccgcgctggaggagatgcgcccg

SEQ ID NO: 47 - DNA, 50 bp, synthetic construct
  source 1..50: mol_type = other DNA
cccgcgaatgcgcaccgccagcgtcgagttggtttgcagcctctcgcggg

SEQ ID NO: 48 - DNA, 39 bp, synthetic construct
  source 1..39: mol_type = other DNA
tggcgcgggtaaccggtggcgccgcgctggcccgcgcca

SEQ ID NO: 49 - DNA, 45 bp, synthetic construct
  source 1..45: mol_type = other DNA
ccgcgccggactaccatcacgtcgtggcaaccaaggactgcgcgg

SEQ ID NO: 50 - DNA, 45 bp, synthetic construct; identical to SEQ ID NO: 34
  source 1..45: mol_type = other DNA

SEQ ID NO: 51 - DNA, 46 bp, synthetic construct; identical to SEQ ID NO: 36
  source 1..46: mol_type = other DNA

SEQ ID NO: 52 - DNA, 44 bp, synthetic construct
  source 1..44: mol_type = other DNA
cgcggcgtgttctacaagggagcctacaccggagcgtagccgcg

SEQ ID NO: 53 - DNA, 45 bp, synthetic construct; identical to SEQ ID NO: 43
  source 1..45: mol_type = other DNA

SEQ ID NO: 54 - DNA, 41 bp, synthetic construct; identical to SEQ ID NO: 45
  source 1..41: mol_type = other DNA

SEQ ID NO: 55 - DNA, 44 bp, synthetic construct; identical to SEQ ID NO: 46
  source 1..44: mol_type = other DNA

SEQ ID NO: 56 - DNA, 44 bp, synthetic construct
  source 1..44: mol_type = other DNA
cggcgcggagccaccggttaccgccagcgagccaccctgcgccg

SEQ ID NO: 57 - DNA, 46 bp, synthetic construct
  source 1..46: mol_type = other DNA
cgcgcggagccaccctcgcataagtcgttctgcacgtcgacgcgcg

SEQ ID NO: 58 - DNA, 43 bp, synthetic construct
  source 1..43: mol_type = other DNA
cgcgcgtcgatgatgatcaacgcccgcatacgtccaccgcgcg

SEQ ID NO: 59 - DNA, 44 bp, synthetic construct
  source 1..44: mol_type = other DNA
accgcgaagtccttggttgccacgacgtgatggtagtccgcggt

SEQ ID NO: 60 - DNA, 40 bp, synthetic construct
  source 1..40: mol_type = other DNA
tcagcgacgtagtccgctgcttcggccaggtagtcgctga

SEQ ID NO: 61 - DNA, 33 bp, synthetic construct
  source 1..33: mol_type = other DNA
gtcgctgatggcgcgggccagcgcggcgccacc

SEQ ID NO: 62 - DNA, 44 bp, synthetic construct
  source 1..44: mol_type = other DNA
cgcgctgacgcaatgcggtggccacgacgaggaatagtagcgcg

SEQ ID NO: 63 - DNA, 42 bp, synthetic construct
  source 1..42: mol_type = other DNA
caggcgaatagtccagtgtgccggagaagtggtcaccgcctg

SEQ ID NO: 64 - DNA, 45 bp, synthetic construct
  source 1..45: mol_type = other DNA
cgccgaggtcacccgggtcgatgtggaagtccttggttgtcggcg

SEQ ID NO: 65 - DNA, 44 bp, synthetic construct
  source 1..44: mol_type = other DNA
ctgcgcgcacccttgtagaacaccgcctcgattgccgagcgcag

SEQ ID NO: 66 - DNA, 39 bp, synthetic construct
  source 1..39: mol_type = other DNA
ccgctcgattgccgacgtgtccagactgggatggagcgg

SEQ ID NO: 67 - DNA, 40 bp, synthetic construct
  source 1..40: mol_type = other DNA
cgccgagtccgcgccgggagtaccgctgacgcaatcggcg

SEQ ID NO: 68 - DNA, 41 bp, synthetic construct
  source 1..41: mol_type = other DNA
cgcgcattcagcagtggcgtgccgttctcgtcgactgcgcg

SEQ ID NO: 69 - DNA, 40 bp, synthetic construct
  source 1..40: mol_type = other DNA
cgcgcgtcgactccttagaagccgctgtacgctccgcgcg

SEQ ID NO: 70 - DNA, 42 bp, synthetic construct
  source 1..42: mol_type = other DNA
cctgcacggcaacgcggcgtagatgaggtcgatgtgtgcagg

SEQ ID NO: 71 - DNA, 42 bp, synthetic construct
  source 1..42: mol_type = other DNA
cgcggatcgatgtggtaggtattgcaaccgatcatttccgcg

SEQ ID NO: 72 - DNA, 43 bp, synthetic construct
  source 1..43: mol_type = other DNA
cgtggattgcgtaccgcgtcctcggccgtctggcgcacccacg

SEQ ID NO: 73 - DNA, 41 bp, synthetic construct
  source 1..41: mol_type = other DNA
cccagacacaatgatcggtggcaataccgaccacatctggg

SEQ ID NO: 74 - DNA, 32 bp, synthetic construct
  source 1..32: mol_type = other DNA
ctcgatgctggcggtgcgcatctcctccagcg

SEQ ID NO: 75 - DNA, 43 bp, synthetic construct
  source 1..43: mol_type = other DNA
cgcgctgcgacggtagtatcggccgacacaccagctgagcgcg

SEQ ID NO: 76 - DNA, 42 bp, synthetic construct
  source 1..42: mol_type = other DNA
ccgtgccgacacacccgctgtcaggtccaccagcacgcacgg

SEQ ID NO: 77 - DNA, 45 bp, synthetic construct
  source 1..45: mol_type = other DNA
cggcgcgtcgatgatgatcaacgcccgcatacgtccaccgcgccg

SEQ ID NO: 78 - DNA, 44 bp, synthetic construct
  source 1..44: mol_type = other DNA
cgcgctgacgcaatgcggtggccacgacgaagaatagtagcgcg

SEQ ID NO: 79 - DNA, 42 bp, synthetic construct
  source 1..42: mol_type = other DNA
acggcgaatagtccgttgtgccggagaagtggtcaccgccgt

SEQ ID NO: 80 - DNA, 40 bp, synthetic construct
  source 1..40: mol_type = other DNA
cgcgcgtcgactccttcgaagccgctgtacgctccgcgcg

SEQ ID NO: 81 - DNA, 42 bp, synthetic construct
  source 1..42: mol_type = other DNA
cgcgagtcgatgtggtaggtattgcgaccgatcattctcgcg

SEQ ID NO: 82 - DNA, 41 bp, synthetic construct
  source 1..41: mol_type = other DNA
ccgcgacacaatgatcgatggcaataccgaccacatcgcgg

SEQ ID NO: 83 - DNA, 27 bp, synthetic construct
  source 1..27: mol_type = other DNA
ccgcaactattcttcgtcgtggtgcgg

SEQ ID NO: 84 - DNA, 35 bp, synthetic construct
  source 1..35: mol_type = other DNA
ccgagcacaccggactattcctcgtcgtggctcgg

SEQ ID NO: 85 - DNA, 35 bp, synthetic construct
  source 1..35: mol_type = other DNA
ccgagccacgacgaagaatagtccggtgtgctcgg

SEQ ID NO: 86 - DNA, 27 bp, synthetic construct
  source 1..27: mol_type = other DNA
ccgagactattcttcgtcgtggctcgg

SEQ ID NO: 87 - DNA, 29 bp, synthetic construct
  source 1..29: mol_type = other DNA
ccagcgactattcttcgtcgtggcgctgg

SEQ ID NO: 88 - DNA, 28 bp, synthetic construct
  source 1..28: mol_type = other DNA
ccgcaccggactattcctcgtcgtgcgg

SEQ ID NO: 89 - DNA, 28 bp, synthetic construct
  source 1..28: mol_type = other DNA
ccgcacgacgaagaatagtccggtgcgg

(19) United States
(12) Patent Application Publication
(10) Pub. No.: US 2026/0242878 A1
(43) Pub. Date: Aug. 20, 2026

(54) METHODS AND SYSTEMS FOR DETECTING PYRAZINAMIDE RESISTANCE MUTATIONS IN MYCOBACTERIUM TUBERCULOSIS

(71) Applicant: Rutgers, The State University of New Jersey, New Brunswick, NJ (US)

(72) Inventors: David Alland, Bernardsville, NJ (US); Soumitesh Chakravorty, Santa Clara, CA (US)

(73) Assignee: Rutgers, The State University of New Jersey, New Brunswick, NJ (US)

(21) Appl. No.: 19/489,526

(22) PCT Filed: Jun. 3, 2024
(86) PCT No.: PCT/US2024/032255

§ 371 Date: Dec. 2, 2025

Related U.S. Application Data
(60) Provisional application No. 63/506,227, filed on Jun. 5, 2023.

(51) Int. Cl. C12Q 1/689; C12Q 1/6827

(52) U.S. Cl. CPC C12Q 1/689; C12Q 1/6827; C12Q 2600/106; C12Q 2600/156; C12Q 2600/16

(57) ABSTRACT

This disclosure provides an improved assay capable of rapidly detecting pyrazinamide resistance mutations in Mycobacterium tuberculosis.

### REFERENCE TO A SEQUENCE LISTING

This application incorporates by reference the Sequence Listing submitted in Computer Readable Form as a xml file named “Sequence listing 096738.00779.xml” created on May 28, 2024 and containing 80,737 bytes.

### CROSS-REFERENCE TO RELATED APPLICATIONS

This application claims priority under 35 U.S.C. § 119 (e) to U.S. Provisional Patent Application No. 63/506,227, filed Jun. 5, 2023. The foregoing application is incorporated by reference herein in its entirety.

### FIELD OF THE INVENTION

This invention relates to methods and systems for rapid detection of pyrazinamide resistance mutations in Mycobacterium tuberculosis.

### BACKGROUND OF THE INVENTION

Tuberculosis is a respiratory disease caused by infection with Mycobacterium tuberculosis. Pyrazinamide (PZA) is a prodrug whose inclusion into first line tuberculosis (TB) therapy has been key to shortening TB treatment to six months, likely through its ability to sterilize persistent populations of bacteria in necrotic lesions. Pyrazinamide has also been a key component of many multidrug-resistant (MDR) TB treatment regimens and is a critical component of the newly described short-course treatment for drug-susceptible TB (INH, high dose rifapentine, moxifloxacin and pyrazinamide), the first four-month TB treatment approved by the U.S. Centers for Disease Control (CDC). Numerous mechanistic studies have provided contradictory evidence on how pyrazinamide acts, suggesting that it either inhibits fatty acid biosynthesis or pantothenate biosynthesis, or that it has an effect on trans-translation. However, it is clear that the conversion of pyrazinamide to pyrazinoic acid (POA) is essential to pyrazinamide's activity. The efficacy of pyrazinamide has been substantially compromised by the development of pyrazinamide resistance in approximately half of MDR TB cases. Pyrazinamide is activated by pyrazinamidase (PZase or PncA), which is an amidase that hydrolyzes pyrazinamide into its active moiety, pyrazinoic acid. Approximately 95% of all clinical resistance to pyrazinamide results from the development of mutations in the Mycobacterium tuberculosis pncA gene. The need for improved pyrazinamide performance has become urgent due to the ever-increasing global incidence of MDR and extensively drug-resistant (XDR) TB and the critical role of pyrazinamide in short-course TB treatment.

Pyrazinamide resistance mutations in the pncA gene are scattered widely throughout the entire 561 bp region of the pncA gene, as well as in a part of the pncA promoter region with >500 different pyrazinamide associated mutations reported so far, making it difficult to detect pyrazinamide resistance. Molecular detection of pyrazinamide resistance has been largely restricted to DNA sequencing since resistance has been associated with hundreds of different mutations in the pncA gene. A line probe assay and a molecular beacon (MB) based “lights on/lights off” approach have provided proof of principle evidence that probes which tile the entire pncA gene can detect pyrazinamide resistance, with 98.9% sensitivity and 91.8% specificity compared to phenotypic resistance testing, by identifying any sequence which varies from the wild type (WT) pncA gene reference. However, line probe assays require access to a highly technical laboratory, and the lights on/lights off approach (while related to sloppy molecular beacon (SMB) melting temperature (Tm) analysis) is not sufficiently sensitive or robust for real world use.

### SUMMARY OF THE INVENTION

This disclosure addresses the need mentioned above in a number of aspects. In one aspect, this disclosure provides a method for identifying one or more pyrazinamide-resistant mutations in a pncA gene of Mycobacterium tuberculosis.

In some embodiments, the method comprises: (a) amplifying a nucleic acid of Mycobacterium tuberculosis in a sample with one or more primer pairs to obtain one or more amplicons, wherein each of the one or more primer pairs comprises a forward primer and a reverse primer, wherein the one or more primer pairs is each specific for a target region of the nucleic acid, and wherein the one or more amplicons respectively correspond to one or more target regions of the nucleic acid; (b) contacting the one or more amplicons with one or more probes under a condition conducive to a hybridization reaction to form one or more probe-amplicon hybrids; (c) determining a melting temperature (Tm) of each of the one or more probe-amplicon hybrids; (d) determining a difference between the melting temperature of each of the one or more probe-amplicon hybrids and a reference melting temperature corresponding to the same probe-amplicon hybrid; and (e) identifying one or more pyrazinamide-resistant mutations in the nucleic acid in the sample based on the difference between the melting temperature of each of the one or more probe-amplicon hybrids and the reference melting temperature corresponding to the same probe-amplicon hybrid.

In some embodiments, the one or more primer pairs are adapted to amplify an antisense strand of the nucleic acid. In some embodiments, the one or more probes hybridize to the antisense strand of the one or more amplicons.

In some embodiments, a mismatch caused by the one or more mutations within the one or more probe-amplicon hybrids is located at the center of the one or more probes. In some embodiments, the difference is equal to or greater than 1 degree Celsius. In some embodiments, at least one of the one or more target regions comprises one or more mutations that confer pyrazinamide resistance of Mycobacterium tuberculosis.

In some embodiments, the one or more primer pairs comprise six primer pairs. In some embodiments, the one or more probes comprise 1 to 10 probes for each target region.

In some embodiments, the step of amplifying is performed by a polymerase chain reaction (PCR). In some embodiments, the step of amplifying may be performed by an asymmetric PCR.

In some embodiments, the step of amplifying for each of the one or more primer pairs is performed in separate reaction mixtures.

In some embodiments, the sample comprises a genomic DNA or fragment thereof of Mycobacterium tuberculosis. In some embodiments, the method comprises extracting the genomic DNA or fragment thereof from the sample.

In some embodiments, the one or more primer pairs comprise a primer having a nucleotide sequence having at least 90% sequence identity with a nucleotide sequence of SEQ ID NOs: 1-25, or having a nucleotide sequence of SEQ ID NOs: 1-25.

In some embodiments, the one or more primer pairs comprise a forward and reverse primer sequence pair set forth respectively in SEQ ID NOs: 1-2; 1-3; 4-5; 6-7; 6-8; 9-10; 11-12; 11-13; 14-15; 16-17; 16-18; 19-20; 21-22; 23-24; and 23-25.

In some embodiments, the one or more probes comprise a probe having a nucleotide sequence having at least 90% sequence identity with a nucleotide sequence of SEQ ID NOs: 26-89, or a nucleotide sequence of SEQ ID NOs: 26-89.

In some embodiments, the one or more probes comprise one or more labels. In some embodiments, the one or more labels comprise at least one of a fluorophore and a quencher. In some embodiments, the one or more labels are located internally or at a terminus of the one or more probes. In some embodiments, the fluorophore is selected from fluorescein, cyanine 3, cyanine 5, TexasRed, and TAMRA. In some embodiments, the quencher is selected from DDQ-I, Dabcyl, Eclipse, Iowa Black FQ, BHQ-1, QSY-7, BHQ-2, DDQ-II, Iowa Black RQ, QSY-21, BHQ-3, IRDye QC-1, ZEN, IBFQ, BHQ1, BHQ2, IBRQ, ZEN, and Licor IRDye QC-1.

In another aspect, this disclosure provides an isolated nucleic acid for identifying one or more pyrazinamide-resistant mutations in the pncA gene of Mycobacterium tuberculosis. In some embodiments, the nucleic acid comprises a nucleotide sequence of SEQ ID NOs: 1-89 or comprising a nucleotide sequence having at least 90% sequence identity with a nucleotide sequence of SEQ ID NOs: 1-89.

In some embodiments, the isolated nucleic acid comprises a nucleotide sequence having at least 90% sequence identity with a nucleotide sequence of SEQ ID NOs: 1-25, or having a nucleotide sequence of SEQ ID NOs: 1-25.

In some embodiments, the isolated nucleic acid comprises a nucleotide sequence having at least 90% sequence identity with a nucleotide sequence of SEQ ID NOs: 26-89, or having a nucleotide sequence of SEQ ID NOs: 26-89.

In some embodiments, the probe comprises one or more labels. In some embodiments, the one or more labels comprise at least one of a fluorophore and a quencher. In some embodiments, the one or more labels are located internally or at a terminus of the probe.

In some embodiments, the fluorophore is selected from fluorescein, cyanine 3, cyanine 5, TexasRed, and TAMRA. In some embodiments, the quencher is selected from BHQ1, BHQ2, and DABCYL.

In yet another aspect, this disclosure further provides a kit comprising the isolated nucleic acid described herein. In some embodiments, the kit comprises a primer pair comprising a forward and reverse primer sequence pair set forth respectively in SEQ ID NOs: 1-2; 1-3; 4-5; 6-7; 6-8; 9-10; 11-12; 11-13; 14-15; 16-17; 16-18; 19-20; 21-22; 23-24; and 23-25.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a map of the existing pncA open reading frame (ORF) and promoter region assay. The position of potential primer (arrows) and sloppy molecular beacon (SMB) probe (lines) binding sites for the pyrazinamide assay are shown. Primers for each amplicon are shown. SMBs that probe each amplicon are coded in the same grey gradient. The pncA ORF is shown, and known pncA mutations are shown as vertical lines. The numbers indicate the codons in the ORF.

FIG. 2 shows the delta Tm values detecting a wide range of mutations in the promoter region and the ORF of the pncA gene (positions-11 to codon 170). The nucleotide and amino acid changes are shown in the first column along the promoter region and different pncA codons respectively. Delta Tm values detecting the mutations are shown along with the amplicons which target the mutation. Negative delta Tm values indicate that the mutant Tm is higher than the reference wild type Tm. RC indicates the reverse complement amplicon. Delta Tm values <1° C. are not shown.

FIG. 3 shows a melting temperature profile of the wild type (WT) and mutant sequences showing clear Tm shifts from the reference WT Tm due to mutations in different regions of the pncA gene.

### DETAILED DESCRIPTION OF THE INVENTION

This disclosure provides nucleic acids, reagents, and methods for detecting pyrazinamide (PZA)-resistant Mycobacterium tuberculosis. The increasing incidence of multidrug-resistant (MDR) and extensively drug-resistant (XDR) TB represents a global public health challenge and emphasizes the need for efficient detection of drug resistance mutations in Mycobacterium tuberculosis samples from patients, particularly mutations associated with pyrazinamide. Current methods available to detect pyrazinamide-resistance mutations in Mycobacterium tuberculosis face significant limitations. Line probe assays require sophisticated laboratory equipment that is not readily available worldwide. Furthermore, as more than five hundred different PZA resistance mutations have been reported, specific and sensitive assays that ensure coverage of the entire pncA gene is essential.

Accordingly, in one aspect, this disclosure provides a method for identifying one or more pyrazinamide-resistant mutations in a pncA gene of Mycobacterium tuberculosis. The methods disclosed herein offer several advantages over current pyrazinamide detection methods. Unlike in high resolution Tm analysis detection methods, which detect mutations by recognizing subtle “melt curve variants,” the methods disclosed herein produce clear and consistent Tm “peaks,” with distinct “Tm shifts” in the presence of a mutation in the pncA target region, which enables highly reproducible Tm value identification. The combination of the reverse strand assay with the sense strand assay identifies additional mutations that may be missed by sense strand assays alone, including by detecting G-T mismatches, and allows for a dTm cut-off value of +2° C., which provides 100% sensitivity and 100% specificity.

In some embodiments, the method comprises: (a) amplifying a nucleic acid of Mycobacterium tuberculosis in a sample with one or more primer pairs to obtain one or more amplicons, wherein each of the one or more primer pairs comprises a forward primer and a reverse primer, wherein the one or more primer pairs is each specific for a target region of the nucleic acid, and wherein the one or more amplicons respectively correspond to one or more target regions of the nucleic acid; (b) contacting the one or more amplicons with one or more probes under a condition conducive to a hybridization reaction to form one or more probe-amplicon hybrids; (c) determining a melting temperature (Tm) of each of the one or more probe-amplicon hybrids; (d) determining a difference between the melting temperature of each of the one or more probe-amplicon hybrids and a reference melting temperature corresponding to the same probe-amplicon hybrid; and (e) identifying one or more pyrazinamide-resistant mutations in the nucleic acid in the sample based on the difference between the melting temperature of each of the one or more probe-amplicon hybrids and the reference melting temperature corresponding to the same probe-amplicon hybrid.

In some embodiments, the one or more primer pairs are adapted to amplify an antisense strand of the nucleic acid. In some embodiments, the one or more probes hybridize to the antisense strand of the one or more amplicons.

In some embodiments, a mismatch caused by the one or more mutations within the one or more probe-amplicon hybrids is located at the center of the one or more probes. In some embodiments, the difference is equal to or greater than 1 degree Celsius. In some embodiments, at least one of the one or more target regions comprises one or more mutations that confer pyrazinamide resistance of Mycobacterium tuberculosis.

In some embodiments, the one or more primer pairs comprise 1 to 10 primer pairs (e.g., 1, 2, 3, 4, 5, 6, 7, 8, 9, 10 primer pairs). In some embodiments, the one or more primer pairs comprise 6 primer pairs. In some embodiments, the one or more probes comprise 1 to 10 probes (e.g., 1, 2, 3, 4, 5, 6, 7, 8, 9, 10 probes) for each target region.

In some embodiments, the step of amplifying a nucleic acid of Mycobacterium tuberculosis with one or more primer pairs to obtain one or more amplicons can be performed by amplifying a nucleic acid of Mycobacterium tuberculosis in a single or multiple amplification reactions, which may include at least the following scenarios. In a first example, each primer pair to a single target gene or several different target genes is used separately to amplify the nucleic acid. In other words, amplification of the nucleic acid for respective primer pairs can be carried out in separate reactions. In a second example, a subset of primer pairs can be used to amplify the nucleic acid separately, for example, such that adjoining overlapping amplicons are not used in the same amplification. In a third example, all primer pairs are used to amplify the nucleic acid in a single amplification reaction. In the second and third examples, additional amplicons can be generated and used to probe for mutations.

The term “primer” refers to any nucleic acid that is capable of specifically hybridizing to a complementary nucleic acid molecule and that provides a free 3′ hydroxyl terminus, which can be extended by a nucleic acid polymerase. As used herein, amplification primers are a pair of nucleic acid molecules that can anneal to 5′ or 3′ regions of a gene (plus and minus strands, respectively, or vice-versa) and contain a short region in between. Under appropriate conditions and with appropriate reagents, such primers permit the amplification of a nucleic acid molecule having the nucleotide sequence flanked by the primers. For in situ methods, a cell or tissue sample can be prepared and immobilized on a support, such as a glass slide, and then contacted with a probe that can hybridize to DNA or RNA. Alternative methods for amplifying nucleic acids corresponding to expressed RNA samples include those described in, e.g., U.S. Pat. No. 7,897,750.

As used herein, the term “oligonucleotide” refers to a short polynucleotide, typically less than or equal to 300 nucleotides long (e.g., in the range of 5 and 150, preferably in the range of 10 to 100, more preferably in the range of 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

The term “probe,” as used herein, refers to an oligonucleotide capable of binding to a target nucleic acid of complementary sequence through one or more types of chemical bonds, usually through complementary base pairing, through hydrogen bond formation. Probes may bind target sequences lacking complete complementarity with the probe sequence depending upon the stringency of the hybridization conditions. There may be any number of base pair mismatches, which will interfere with hybridization between the target sequence and the single-stranded nucleic acids described herein. However, if the number of mutations is so great that no hybridization can occur under even the least stringent of hybridization conditions, the sequence is not a complementary target sequence. A probe may be single-stranded or partially single and partially double-stranded. The strandedness of the probe is dictated by the structure, composition, and properties of the target sequence. Probes may be directly labeled or indirectly labeled, such as with biotin to which a streptavidin complex may later bind.

The term “detection probe” refers to an oligonucleotide having a sequence sufficiently complementary to its target sequence to form a probe: target hybrid (e.g., probe: amplicon hybrid) stable for detection under stringent hybridization conditions. A probe is typically a synthetic oligomer that may include bases complementary to a sequence outside of the targeted region, which does not prevent hybridization under stringent hybridization conditions to the target nucleic acid. A sequence non-complementary to the target may be a homopolymer tract (e.g., poly-A or poly-T), promoter sequence, restriction endonuclease recognition sequence, or sequence to confer desired secondary or tertiary structure (e.g., a catalytic site or hairpin structure), or a tag region which may facilitate detection and/or amplification.

“Stable” or “stable for detection” means that the temperature of a reaction mixture is at least 2° C. below the melting temperature (Tm) of a nucleic acid duplex contained in the mixture, more preferably at least 5° C. below the Tm, and even more preferably at least 10° C. below the Tm.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

“Substantially complementary” means that a nucleic acid or oligonucleotide has a sequence containing at least 10 contiguous bases that are at least 80% (e.g., 85%, 90%, 95%, 96%, 97%, 98%, 99%, and 100%) to at least 10 contiguous bases in a target nucleic acid sequence so that the nucleic acid or oligonucleotide can hybridize or anneal to the target nucleic acid sequence under, e.g., the annealing condition of a PCR assay or probe-target hybridization condition. Complementarity between sequences may be expressed a number of base mismatches in each set of at least 10 contiguous bases being compared. The term “substantially identical” means that a first nucleic acid is at least 80% (e.g., 85%, 90%, 95%, 96%, 97%, 98%, 99%, and 100%) complementary to a second nucleic acid so that the first nucleic acid is substantially complementary to and is capable of hybridizing to the complement of the second nucleic acid under PCR annealing or probe-target hybridization conditions.

“Hybridization” or “hybridizing” or “hybridize” or “anneal” refers to the ability of completely or partially complementary nucleic acid strands to come together under specified hybridization conditions in a parallel or preferably antiparallel orientation to form a stable double-stranded structure or region (sometimes called a “hybrid” or “duplex” or “stem”) in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

In some embodiments, modified nucleotide bases (e.g., including, but not limited to side chain alkyl modifications) can be incorporated in the oligonucleotide to enhance the “hybridization” process between the probe and the target, resulting in an increased Tm value of the probe: target hybrid.

In some embodiments, the step of amplifying may be performed by a polymerase chain reaction (PCR). In some embodiments, the step of amplifying may be performed by an asymmetric PCR. In some embodiments, the step of amplifying may be performed using a cDNA generated by reverse transcriptase PCR (RT-PCR) from an RNA molecule.

As used herein, the term “amplification” and its variants include any process for producing multiple copies or complements of at least some portion of a polynucleotide, the polynucleotide typically being referred to as a “template.” The template polynucleotide can be single stranded or double stranded. A template may be a purified or isolated nucleic acid, or may be non-purified or non-isolated. Amplification of a given template can result in the generation of a population of polynucleotide amplification products, collectively referred to as an “amplicon.” The polynucleotides of the amplicon can be single stranded or double stranded, or a mixture of both. Typically, the template will include a target sequence, and the resulting amplicon will include polynucleotides having a sequence that is either substantially identical or substantially complementary to the target sequence. In some embodiments, the polynucleotides of a particular amplicon are substantially identical, or substantially complementary, to each other; alternatively, in some embodiments, the polynucleotides within a given amplicon can have nucleotide sequences that vary from each other. Amplification can proceed in a linear or exponential fashion, and can involve repeated and consecutive replications of a given template to form two or more amplification products. Some typical amplification reactions involve successive and repeated cycles of template-based nucleic acid synthesis, resulting in the formation of a plurality of daughter polynucleotides containing at least some portion of the nucleotide sequence of the template and sharing at least some degree of nucleotide sequence identity (or complementarity) with the template. In some embodiments, each instance of nucleic acid synthesis, which can be referred to as a “cycle” of amplification, includes creating free 3′ end (e.g., by nicking one strand of a dsDNA), thereby generating a primer and primer extension steps; optionally, an additional denaturation step can also be included wherein the template is partially or completely denatured. In some embodiments, one round of amplification includes a given number of repetitions of a single cycle of amplification. For example, a round of amplification can include 5, 10, 15, 20, 25, 30, 35, 40, 50, or more repetitions of a particular cycle. In one exemplary embodiment, amplification includes any reaction wherein a particular polynucleotide template is subjected to two consecutive cycles of nucleic acid synthesis. The synthesis can include template-dependent nucleic acid synthesis.

As used herein, the term “asymmetric PCR” refers to the preferential PCR amplification of one strand of a DNA target by adjusting the molar concentration of the primers in a primer pair so that they are unequal. An asymmetric PCR assay produces a predominantly single stranded product and a smaller quantity of a double stranded product as a result of the unequal primer concentrations. As asymmetric PCR proceeds, the lower concentration primer is quantitatively incorporated into a double stranded DNA amplicon, but the higher concentration primer continues to prime DNA synthesis, resulting in continued accumulation of a single stranded product. Asymmetric PCR also includes the use of a single primer for amplification using a template or a double stranded amplicon from a previous amplification, with the amplicon including the primer binding site for the single primer.

Amplification may also include isothermal amplification. The term “isothermal” means conducting a reaction at a substantially constant temperature, i.e., without varying the reaction temperature in which a nucleic acid polymerization reaction occurs. Isothermal temperatures for isothermal amplification reactions depend on the strand-displacing nucleic acid polymerase used in the reactions. Generally, the isothermal temperatures are below the melting temperature (Tm; the temperature at which half of the potentially double-stranded molecules in a mixture are in a single-stranded, denatured state) of the predominant reaction product, i.e., generally 90° C. or below, usually between about 20° C. and 75° C., and preferably between about 30° C. and 60° C., or more preferably at about 37° C.

As used herein, the term “contacting” and its variants, when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as a polymerase, a primer set or a probe, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

In some embodiments, the sample comprises a genomic DNA or fragment thereof of Mycobacterium tuberculosis. In some embodiments, the method comprises extracting the genomic DNA or fragment thereof from the sample. In some embodiments, the sample may be obtained from a subject.

As used herein, the term “subject” refers to any organism having a genome, such as a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc.).

As used herein, a “sample” refers to any biological fluid or tissue obtained from an organism (e.g., patient), or a microorganism (e.g., bacteria, virus or fungi) or from components (e.g., blood) of an organism. The sample may be of any biological tissue, cell(s) or fluid. The sample may be a “clinical sample,” which is a sample derived from a subject, such as a human patient or veterinary subject, which may or may not contain an infectious microorganism (bacteria, virus or fungi). Useful biological samples include, without limitation, whole blood, saliva, urine, synovial fluid, bone marrow, cerebrospinal fluid, vaginal mucus, cervical mucus, nasal secretions, sputum, semen, amniotic fluid, bronchoalveolar lavage fluid, and other cellular exudates from a patient or subject. Such samples may further be diluted with saline, buffer or a physiologically acceptable diluent. Alternatively, such samples are concentrated by conventional means. Biological samples may also include sections of tissues, such as frozen sections taken for histological purposes. A biological sample may also be referred to as a “patient sample.” A biological sample may also include a substantially purified or isolated protein, membrane preparation, or cell culture.

As used herein, the term “reference” value (e.g., reference melting temperature) refers to a value that statistically correlates to a particular outcome when compared to an assay result. In some embodiments, the reference value can be determined from statistical analysis that examines the mean of wild type values. The reference value may be a threshold score value or a cutoff score value. Typically, a reference value will be a threshold above (or below) which one outcome is more probable and below which an alternative outcome is more probable.

In some embodiments, a difference of a value or level (e.g., melting temperature) may be a statistically significant difference between the quantities of an analyte present in a sample as compared to a control. For example, a difference may be statistically significant if the measured level of the analyte falls outside of about 1.0, 2.0, 3.0, 4.0, or 5.0 standard deviations of the mean of any control or reference group.

As used herein, the term “threshold value” refers to a point at which an analysis process may change and/or a point at which an action may be triggered. In some embodiments, the threshold value for the aggregated difference of melting temperature is between 1° C. and 10° C. (e.g., 1, 2, 3, 4, 5, 6, 7, 8, 9, or 10° C.).

In some embodiments, the step of amplifying and the step of contacting may be performed in a single reaction mixture. For example, the reverse transcription step and the amplification step may be performed using a QIAGEN One-Step RT-PCR kit (Qiagen cat. No 210212, Hilden, Germany).

In some embodiments, the step of amplifying for each of one or more primer pairs may be performed in separate reaction mixtures. For example, separate reaction mixtures may be prepared for each primer pair, such that detection of individual mutations in the nucleic acid is performed separately.

As used herein, a “target region,” “target nucleic acid sequence,” or “target sequence” refers to a specific sequence that may include all or part of the sequence of a single-stranded nucleic acid. A target sequence may be within a nucleic acid template or within the genome of a cell, which may be any form of single-stranded or double-stranded nucleic acid. A template may be a purified or isolated nucleic acid, or may be non-purified or non-isolated.

In some embodiments, the one or more primer pairs comprise a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NOs: 1-25, or having a nucleotide sequence of SEQ ID NOs: 1-25.

In some embodiments, the one or more primer pairs comprise a forward and reverse primer sequence pair set forth respectively in SEQ ID NOs: 1-2; 1-3; 4-5; 6-7; 6-8; 9-10; 11-12; 11-13; 14-15; 16-17; 16-18; 19-20; 21-22; 23-24; and 23-25.

In some embodiments, one or more amplicons may include a first amplicon comprising the first target region. In some embodiments, one or more amplicons may include a second amplicon comprising the second target region. In some embodiments, one or more amplicons may include a third amplicon comprising the third target region. In some embodiments, one or more amplicons may include a fourth amplicon comprising the fourth target region. In some embodiments, one or more amplicons may include a fifth amplicon comprising the fifth target region. In some embodiments, one or more amplicons may include a sixth amplicon comprising the sixth target region. In some embodiments, one or more amplicons may include a seventh amplicon comprising the seventh target region. In some embodiments, one or more amplicons may include an eighth amplicon comprising the eighth target region. In some embodiments, one or more amplicons may include a nineth amplicon comprising the nineth target region. In some embodiments, one or more amplicons may include a tenth amplicon comprising the tenth target region.

In some embodiments, one or more probes may include a first probe or a second probe capable of hybridizing to the first amplicon. In some embodiments, one or more probes may include a third probe or a fourth probe capable of hybridizing to the second amplicon. In some embodiments, one or more probes may include a fifth probe or a sixth probe capable of hybridizing to the third amplicon.

A probe can be made in various detection formats, such as dual labeled probes, including liner probes, Taqman probes, molecular beacon probes, and sloppy molecular beacon (SMB) probes. A “sloppy” probe refers to a probe that is mismatch-tolerant. Mismatch-tolerant probes hybridize with and generate a detectable signal for more than one target sequence at a detection temperature in an assay, and various hybrids so formed will have different melting temperatures. Linear, or random coil, single-stranded probes are generally mismatch tolerant. Examples of such probes are hairpin or linear probes with an internal fluorescent moiety whose level of fluorescence increases upon hybridization to one or another target strand. See, e.g., U.S. Pat. Nos. 7,662,550 and 5,925,517. US20130095479.

In some embodiments, the sloppy probes are dual-labeled hairpin probes or molecular beacon probes, described in U.S. Pat. Nos. 7,662,550 and 5,925,517. These hairpin probes contain a target binding sequence flanked by a pair of arms complementary to one another. They can be DNA, RNA, or PNA, or a combination of all three nucleic acids. Furthermore, they can contain modified nucleotides and modified internucleotide linkages. They can have a first fluorophore on one arm and a second fluorophore on the other arm, wherein the absorption spectrum of the second fluorophore substantially overlaps the emission spectrum of the first fluorophore. Such hairpin probes may be “molecular beacon probes” that have a fluorophore on one arm and a quencher on the other arm such that the probes are dark when free in solution. They can also be wavelength-shifting molecular beacon probes with, for example, multiple fluorophores on one arm that interact by fluorescence resonance energy transfer (FRET), and a quencher on the other arm. They can also have a first fluorophore on one arm and a second fluorophore on the other arm with an internal quencher molecule in the target binding sequence region. The target binding sequences can be, for example, 12 to 50, or 25 to 50 nucleotides in length, and the hybridizing arms can be 4 to 10 or 4 to 7 (e.g., 5 or 7) nucleotides in length. A portion of the arm sequence of the probe can have complementarity to the target sequence and a portion of the target region of the hairpin probe can participate in forming the hairpin along with the arm sequence. Molecular beacon probes can be tethered to primers, as described in U.S. Pat. Nos. 7,662,550 and 5,925,517 and WO 01/31062.

Sloppy molecular beacon (SMB) probes thus refer to such a class of fluorescently labeled hairpin oligonucleotide hybridization probes. Such probes produce a detectable signal in a homogeneous assay, that is, without having to separate probes hybridized to target unbound probes. By virtue of their ability to bind to more than one variants of a given target sequence, the probes can be used in assays to detect the presence of one variant of a nucleic acid sequence segment of interest from among a number of possible variants or even to detect the presence of two or more variants. The probes can therefore be used in combinations of two or more in the same assay. Because they differ in target binding sequence, their relative avidities for different variants are different. For example, a first probe may bind strongly to a wild-type sequence, moderately to a first allele, weakly to a second allele and not at all to a third allele; while a second probe may bind weakly to the wild-type sequence and the first variant, and moderately to the second variant and the third variant. Additional sloppy probes will exhibit yet different binding patterns due to their different target binding sequences. Thus, the patterns of the fluorescence emission spectra from combinations of sloppy probes can define different microbial strains or species, as well as allelic variants/mutation of genes.

As the sloppy probes reproducibly fluoresce with variable intensities after binding to different DNA sequences, combinations can be used in, for example, rapid, and sensitive nucleic acid amplification reaction assays (e.g., PCR-based assays) that identify multiple pathogens or variants in a single reaction container. It is understood, however, that the assays can also be performed on samples suspected of containing directly detectable amounts of unamplified target nucleic acids. This identification assay is based on analyzing the spectra of a set of partially hybridizing sloppy signaling probes, such as sloppy molecular beacon probes, each labeled with a fluorophore that emits light with a different wavelength optimum, to generate “signature spectra” of species-specific or variant-specific DNA sequences.

Using the probes, multiplexing can be achieved, for example, by designing a different allele-discriminating molecular beacon probe for each target and labeling each probe differentially. (See, e.g., U.S. Pat. Nos. 7,662,550 and 5,925,517, WO 01/31062, and Tyagi et al. (2000) Nature Biotechnology 18:1191-1196). Mixtures of allele-discriminating probes, each comprising aliquots of multiple colors, extend the number of probe signatures. To that end, every molecular beacon-target hybrid with a unique melting temperature will have corresponding unique signal intensity at a defined temperature and concentration of probe and amplicon. Thus, a limited number of sloppy probes could be used as probes to identify many different possible target sequences in a real-time PCR assay. The probes can be added to the amplification reaction mixture before, during, or after the amplification. See U.S. Pat. No. 7,662,550.

In some embodiments, the probes may include one or more labels. As used herein, a “label” or “reporter molecule” is a chemical or biochemical moiety useful for labeling a nucleic acid (including a single nucleotide), polynucleotide, oligonucleotide, or protein ligand, e.g., amino acid or antibody. Examples include fluorescent agents, chemiluminescent agents, chromogenic agents, quenching agents, radionucleotides, enzymes, substrates, cofactors, inhibitors, magnetic particles, and other moieties known in the art. Labels or reporter molecules are capable of generating a measurable signal and may be covalently or noncovalently joined to an oligonucleotide or nucleotide (e.g., a non-natural nucleotide) or ligand.

In some embodiments, the probe comprises one or more labels. In some embodiments, the one or more labels comprise at least one of a fluorophore and a quencher. In some embodiments, the one or more labels are located internally or at a terminus of the probe.

In some embodiments, the labels may include a fluorophore and/or a quencher.

As used herein, a “fluorophore” includes a molecule that is capable of absorbing energy at a wavelength range and releasing energy at a wavelength range other than the absorbance range. In some embodiments, the fluorophore is a molecule that is capable of absorbing energy at about 250 nm to about 900 nm, and can release energy at a wavelength range of about 260 nm to about 910 nm. The term “excitation wavelength” refers to the range of wavelengths at which a fluorophore absorbs energy. The term “emission wavelength” refers to the range of wavelengths that the fluorophore releases energy or fluoresces.

Examples of fluorophores include but are not limited to fluorescein, Texas Red, DAPI, PI, acridine orange, Alexa fluors, e.g., Alexa 350, Alexa 405 or Alexa 488, cyanine dyes such as Cy3, Cy5, and Cy7, coumarin, ethidium bromide, fluorescein, BODIPY, rhodol, Rox, 5-carboxyfluorescein, 6-carboxyfluorescein, an anthracene, 2-amino-4-methoxynapthalene, a phenalenone, an acridone, fluorinated xanthene derivatives, α-naphtol, β-napthol, 1-hydroxypyrene, coumarins, e.g., 7-amino-4-methyl coumarin (AMC) or 7-amino-4-trifluoromethyl coumarin (AFC), rhodamines, e.g., tetramethyl rhodamine, rhodamine-110, carboxyrhodamine, cresyl violet, or resorufin, as well as fluorophores disclosed in U.S. Pat. No. 6,420,130 (Makings, et al.), the disclosure of which is incorporated by reference herein Fluorophores include cyanine dyes, such as compounds of the formula Ar—[CH═CH]ₙ—[CH═]ₘAr, wherein Ar is an aryl or heteroaryl group, n is 1, 2, 3, or 4, m is 0 or 1, and wherein each Ar includes a quaternary nitrogen or a nitrogen capable of being quaternized through resonance Examples of such aryl or heteroaryl groups include dimethyl-aminophenyl, imidazole, pyridine, pyrrole, quinoline, thiazole, and indole, each optionally substituted The fluorophore can be a compound that is inherently fluorescent or demonstrates a change in fluorescence upon binding to a biological compound, i.e., it can be fluorogenic, or its intensity can be diminished by quenching Fluorophores may contain substituents that alter the solubility, spectral properties or physical properties of the fluorophore Various fluorophores are known to those skilled in the art and also include, but are not limited to benzofurans, quinolines, quinazolinones, indoles, benzenols, borapolyazaindacene, and xanthenes including fluorescein, rhodamine, and rhodol, as well as other fluorophores described in Richard P Haugland's The Handbook. A Guide to Fluorescent Probes and Labeling Technologies (10^{th} edition, 2005), which describes numerous fluorophores available from Invitrogen Molecular Probes

Examples of quenchers may include, but are not limited to, DDQ-I, Dabcyl, Eclipse, Iowa Black FQ, BHQ-1, QSY-7, BHQ-2, DDQ-II, Iowa Black RQ, QSY-21, BHQ-3, IRDye QC-1, ZEN, IBFQ, BHQ1, BHQ2, IBRQ, ZEN, and Licor IRDye QC-1.

In some embodiments, the one or more probes comprise a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NOs: 26-89, a nucleotide sequence of SEQ ID NOs: 26-89.

In another aspect, this disclosure provides an isolated nucleic acid for identifying one or more pyrazinamide-resistant mutations in a pncA gene and a part of the promoter region of Mycobacterium tuberculosis. In some embodiments, the nucleic acid comprises a nucleotide sequence of SEQ ID NOs: 1-89 or comprising a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NOs: 1-89.

In some embodiments, the isolated nucleic acid comprises a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NOs: 1-25, a nucleotide sequence of SEQ ID NOs: 1-25.

In some embodiments, the isolated nucleic acid comprises a nucleotide sequence having at least 80% (e.g., 80%, 82%, 84%, 86%, 88%, 90%, 91%, 92%, 93%, 94%, 95%, 96%, 97%, 98%, 99%) sequence identity with a nucleotide sequence of SEQ ID NOs: 26-89, a nucleotide sequence of SEQ ID NOs: 26-89.

In yet another aspect, this disclosure provides a kit comprising the isolated nucleic acid as disclosed herein. In some embodiments, the kit may include reagents for performing the above-described methods, including PCR and/or probe-target (e.g., probe-amplicon) hybridization reactions. To that end, one or more of the reaction components, e.g., PCR primers, polymerase, and probes, for the methods disclosed herein can be supplied in the form of a kit for use. In such a kit, an appropriate amount of one or more reaction components is provided in one or more containers or held on a substrate.

In yet another aspect, this disclosure further provides a kit comprising the isolated nucleic acid described herein.

In some embodiments, the kit may include a primer pair comprising a forward and reverse primer sequence pair set forth respectively in SEQ ID NOs: 1-2; 1-3; 4-5; 6-7; 6-8; 9-10; 11-12; 11-13; 14-15; 16-17; 16-18; 19-20; 21-22; 23-24; and 23-25. The kit also contains additional materials for practicing the above-described methods. In some embodiments, the kit contains some or all of the reagents and materials for performing a method that uses primers and/or probes according to this disclosure. Some or all of the components of the kits can be provided in containers separate from the container(s) containing the primers and/or probes of this disclosure. Examples of additional components of the kits include, but are not limited to, one or more different polymerases, one or more control reagents (e.g., probes or PCR primers or control templates), and buffers for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, probes and/or primers) can be suspended in an aqueous solution or as a freeze-dried or lyophilized powder, pellet, or bead. In the latter case, the components, when reconstituted, form a complete mixture of components for use in an assay. The kits can be provided at any suitable temperature. For example, for storage of kits containing protein components (e.g., an enzyme) in a liquid, it is preferred that they are provided and maintained below 0° C., preferably at or below −20° C., or otherwise in a frozen state.

A kit or system of this disclosure may contain, in an amount sufficient for at least one assay, any combination of the components described herein. In some applications, one or more reaction components may be provided in pre-measured single use amounts in individual, typically disposable, tubes or equivalent containers. With such an arrangement, a PCR assay can be performed by adding a target nucleic acid or a sample/cell containing the target nucleic acid to the individual tubes directly. The amount of a component supplied in the kit can be any appropriate amount, and may depend on the target market to which the product is directed. In some embodiments, the kit may include PCR nano-beads or reagents to perform liquid assays.

The container(s) in which the components are supplied can be any conventional container that is capable of holding the supplied form, for instance, microfuge tubes, ampoules, bottles, or integral testing devices, such as fluidic devices, cartridges, lateral flow, or other similar devices.

The kits can also include packaging materials for holding the container or a combination of containers. Typical packaging materials for such kits and systems include solid matrices (e.g., glass, plastic, paper, foil, micro-particles, and the like) that hold the reaction components or detection probes in any of a variety of configurations (e.g., in a vial, microtiter plate well, microarray, and the like). The kits may further include instructions recorded in a tangible form for use of the components.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

“Nucleic acid” or “oligonucleotide” or “polynucleotide” as used herein refers to at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single-stranded or double-stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods. Nucleic acids may contain modified deoxyribo- and ribo-nucleotide bases with side chain alkyl modifications like propynyl modification.

A “nucleic acid duplex,” “duplex,” “stem,” “nucleic acid hybrid,” or “hybrid” refers to a stable nucleic acid structure comprising a double-stranded, hydrogen-bonded region, e.g., RNA: RNA, RNA: DNA, and DNA: DNA duplex molecules and analogs thereof. Such structure may be detected by any known means, e.g., by using a labeled probe, an optically active probe-coated substrate sensitive to changes in mass at its surface (U.S. Pat. No. 6,060,237), or binding agents (U.S. Pat. No. 5,994,056).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” a target includes determining the amount of the target present, as well as determining whether it is present or absent.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal or within a unicellular organism like a microbe.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of this disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

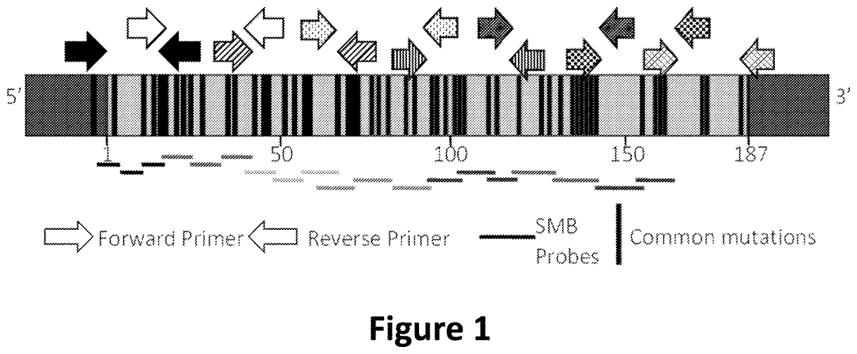

A series of molecular probes were used to tile the entire pncA gene and a part of its promoter region and to detect all mutations responsible for pyrazinamide (PZA) resistance while distinguishing these mutants from rare variants that have “neutral” pncA polymorphisms that do not encode for resistance. Eight overlapping asymmetric PCR assays were developed to query the entire target region in the pncA gene which can harbor pyrazinamide resistance inducing mutations. Each individual assay targets a specific region of the pncA gene, including a part of the promoter, and uses 3-4 probes to query the mutations (see FIG. 1). The assay was tested on a panel of clinical DNA samples harboring mutations throughout the pncA gene and the promoter region. In addition, a “mirror image” reverse strand assay was also developed to selectively amplify the complementary strand to ensure detection of the mutations which were missed in the sense strand assay due to lack of substantial mutation induced Tm shift when compared to the wild type Tm. The first-generation sense strand assay was validated on a panel of 39 clinical DNA samples with 39 different mutations including a silent mutation in codon 65.

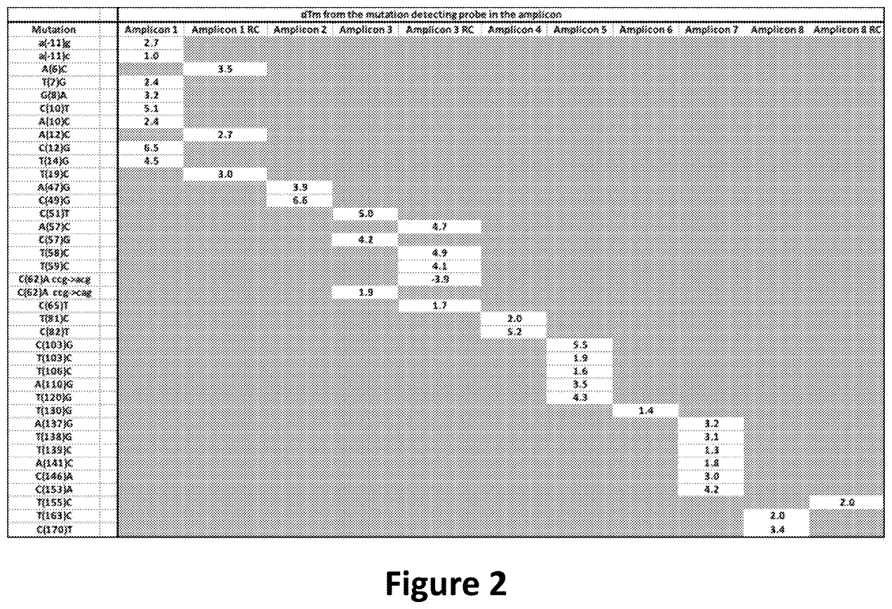

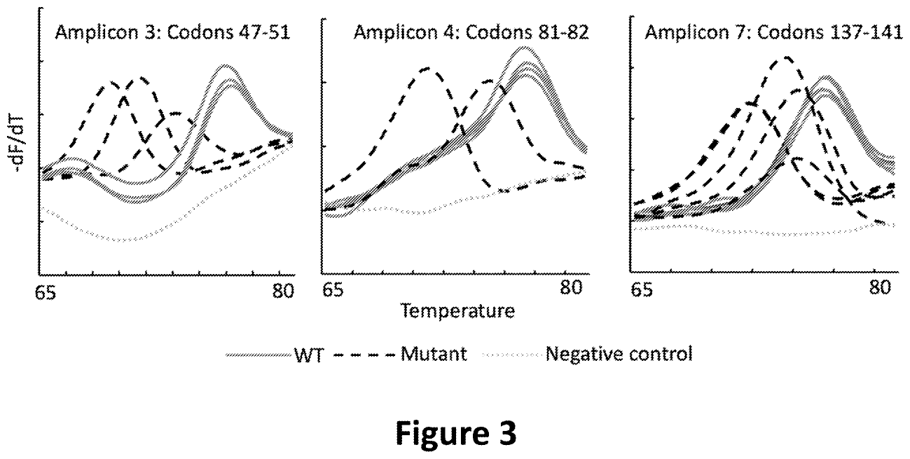

It was observed that the assay worked remarkably well and identified 34 of the 39 mutations tested, showing a sensitivity of detection of 87% (FIGS. 2 and 3). Thirty DNA samples from pan-susceptible laboratory and clinical strains were also tested, and all the samples were identified as wild type (WT) by the assay showing a specificity of 100%. When the mutations that were missed by the sense strand assay were analyzed, it was found that ⅗ mutations were missed either because they were outside the queried region of the sense strand probes, or they were at the edge of the probes that did not generate a sufficient Tm difference (dTm) between the WT and the mutant sequences. The remaining two mutations were missed because of a G-T pair between the probe and the target which generated dTm values of less than 1° C. which was below our designated dTm cut-off value for mutation detection. These results were not surprising considering the fact that the probes were designed to detect the wild type sequences as it is impossible to ascertain the position and nature of mutations which can be present anywhere in a ~602 bp DNA stretch including a part of the promoter region. To circumvent this, a reverse strand assay was designed so that the mutations with dTm values less than or equal to +1° C. are at the center of the probes, to ensure definitive detection by increasing their dTm. This strategy also ensured that the G-T mismatches are now detected unequivocally since they change to C-A mismatches in the reverse strand, which generates greater dTm values. This strategy also enabled an increase in the dTm cut off from ±1° C. to #2° C. Combining the sense and the reverse strands assays to detect pncA mutations enabled detection of all the 39/39 mutations in the tested mutation challenge panel, resulting in a sensitivity of 100% and a specificity of 100%. Additionally, molecular beacon probes to specifically detect polymorphic or silent mutations which do not contribute to drug resistance were also designed and included in the assay.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 1
Representative Primers

| SEQ ID | | |
|---|---|---|
| NO | Name | Sequence |
| 1 | pncA-P1 | ggcaaactgcccgggcagtcgc |
| 2 | pncA-tR1 | gttgttgatggtgcgggccagc |
| 3 | pncA-t2R1 | gttgttgatggtgtgggccagc |
| 4 | pncAF3 | cggcggactaccatcacgtcgtg |
| 5 | pncA-tR3 | tggaagttcgtgccgggagtac |
| 6 | pncA1-F | ggcaaactgcccgggcagtc |
| 7 | pncA1-R | gcgcgggccagcgcggcgcc |
| 8 | pncA1-R2 | gtcgctgatggcgcgggccagc |
| 9 | pncA2-F | tgcgagggtggctcgctggcg |
| 10 | pncA2-R | tggtcacccgggtcgatgtgg |
| 11 | pncA3-F | cggcggactaccatcacgtcgtg |
| 12 | pncA3-R | tggaagtccgcgccgggagt |
| 13 | pncA3-R2 | tggaagttcgtgccgggagt |
| 14 | pncA-4F | gccaccgcattgcgtcagcgg |
| 15 | pncA-4R | ttctcgtcgactccttcgaag |
| 16 | pncA-5F | cgtcggcaatcgaggcggtgt |
| 17 | pncA-5R | acatcgacctcatcgacgccg |
| 18 | pncA-5R2T | atatcgacctcatcgacgccg |
| 19 | pncA-6F | cttcgaaggagtcgacgagaa |
| 20 | pncA-6R | gtcctcggccgtctggcgcac |
| 21 | pncA-7F | gcggcaacgcggcgtcgatgag |
| 22 | pncA-7R | cacacccgctgtcaggtccac |
| 23 | pncA-8F | atggcttggccaccagggtgct |
| 24 | pncA-8R | cggttcggcggtgccatcagg |
| 25 | pncA-8R2 | tggtttggtggtgtcatcagg |

TABLE 2
Representative probes (sense probes)

| | | | | SED ID |
|---|---|---|---|---|
| Amplicon | Name | Sequence | | NO |
| Amp1 | pncA- | ccgcgcgaacgtatggtggacgtatgcgggcgttgatcagcgcgg | | 26 |
| | Amp1P1 | | | |
| | pncA- | cgccgctcgtcgacgtgcagaacgacttctgcgagggtgcggcg | | 27 |
| | Amp1P2 | | | |
| | pncA- | gcgagggtggctcgct(txr)ggcggtaaccggtggcgcc | | 28 |
| | Amp1P3 | | | |
| Amp2 | pncA2-P1 | ccggcaggtaaccggtggcgccgcgctggcccgctcaactgccgg | | 29 |
| | pncA2-P2 | ccccgcgccatcagcgactacctgaccgaagcgggg | | 30 |
| | pncA2-P3- | ccgcgtcggactaccatcacgtcgtggcaaccaaggactacgcgg | | 31 |
| | ATS | | | |
| Amp3 | pncA- | cacccggcaaccaaggacttccacatcgacccgggtg | | 32 |
| | Amp3P1 | | | |
| | pncA- | tcgcgacccgggtgaccacttctccggcacaccggactcgcgg | | 33 |
| | Amp3P2 | | | |
| | pncA- | cgcgcgtattcctcgtcgtggccaccgcattgcgtcagccgcgcg | | 34 |
| | Amp3P3 | | | |
| Amp4 | FAM-pncA- | cctgagcgtactcccggcgcggacttccatcccagtcgctcagg | | 35 |
| | Amp4P1 | | | |
| | Cy5-pncA- | cgcgcccccagtctggacacgtcggcaatcgaggcggtgtggcgcg | | 36 |
| | Amp4P2 | | | |
| | pncA- | cgcggcgtgttctacaagggtgcctacaccggagcgtagccgcg | | 37 |
| | Amp4P3- | | | |
| | TxR | | | |
| Amp5 | pncA5-P1- | cggcgctacaagggtgcctacaccggagcgtacagcggcgccg | | 38 |
| | Cy5 | | | |
| | pncA5-P2- | ccgggcttcgaaggagtcgacgagaacggcacgcccgg | | 39 |
| | TxR | | | |
| | pncA5-P3- | ccggacgccactgctgaattggctgcggcaacgtccgg | | 40 |
| | FAM | | | |
| Amp6 | pncA6-P1- | cggccccgaccacatcgacctcatcgacgccgcgttgggccg | | 41 |
| | FAM | | | |
| | pncA6-P2- | gccgctggtggcaataccgatcacatcgacctcatcgacagcggc | | 42 |
| | TxR | | | |
| Amp7 | pncA7- P1- | cgcgcgccaccgatcattgtgtgcgccagacggccgaggagcgcg | | 43 |
| | TxR | | | |
| | pncA7-P2- | cgcggacgcggttcgcaatggcttggccaccagggtgctgtccgcg | | 44 |
| | Cy5 | | | |
| Amp8 | pncA8-P1- | ccgcgctgctggtggacctgacagcgggtgtgtcggcgcgg | | 45 |
| | TxR | | | |
| | pncA8-P2- | cgggcgccgataccaccgtcgccgcgctggaggagatgcgcccg | | 46 |
| | Cy5 | | | |
| | pncA8-P3- | cccgcgaatgcgcaccgccagcgtcgagttggtttgcagcctctcgcggg | | 47 |
| | FAM | | | |
| Amp1 | pncA2-P1sh | tggcgcgggtaaccggtggcgccgcgctggcccgcgcca | | 48 |
| Amp2 | pncA2-P3- | ccgcgccggactaccatcacgtcgtggcaaccaaggactgcgcgg | | 49 |
| | GCS | | | |
| Amp3 | pncA- | cgcgcgtattccucgucguggccaccgcatugcgtcagccgcgcg | | 50 |
| | Amp3P3dU | | | |
| Amp4 | pncA- | cgcgcccccagtcuggacacgtcggcaatcgaggcggtgtggcgcg | | 51 |
| | Amp4P2dU | | | |
| Amp4 | pncA- | cgcggcgtgttctacaagggagcctacaccggagcgtagccgcg | | 52 |
| | Amp4P3ggA- | | | |
| | TxR | | | |
| Amp7 | pncA7- | cgcgcgccaccgatcattgtgugcgccagacggccgaggagcgcg | | 53 |
| | PldU-TxR | | | |
| | pncA8-P1dU- | ccgcgctgctggtggaccugacagcgggtgtgtcggcgcgg | | 54 |
| | TxR | | | |
| | pncA8-P2dU- | cgggcgccgataccaccgtcgccgcgcuggaggagatgcgcccg | | 55 |
| | Cy5 | | | |
| | | | Current | SEQ ID |
| | Name | Sequence | Fluorophore | NO |
| Amp1 | pncA1-RSA- | cggcgcggagccaccggttaccgccagcgagccacc | FAM | 56 |
| | P1 | ctgcgccg | | |
| | pncA1-RSA- | cgcgcggagccaccctcgcataagtcgttctgcacgtc | Cy5 | 57 |
| | P2 | gacgcgcg | | |
| | pncA1-RSA- | cgcgcgtcgatgatgatcaacgcccgcatacgtccacc | TxR | 58 |
| | P3B | gcgcg | | |
| Amp2 | pncA-RC- | accgcgaagtccttggttgccacgacgtgatggtagtc | Cy5 | 59 |
| | A2P1 | cgcggt | | |
| | pncA-RC- | tcagcgacgtagtccgctgcttcggccaggtagtcgct | FAM | 60 |
| | A2P2 | ga | | |
| | pncA-RC- | gtcgctgat(txr)ggcgcgggccagcgcggcgccac | TxR internal | 61 |
| | A2P3 | C | | |
| Amp3 | pncA-RC- | cgcgctgacgcaatgcggtggccacgacgaggaata | TxR | 62 |
| | A3P1 | gtagcgcg | | |
| | pncA-RC- | caggcgaatagtccagtgtgccggagaagtggtcacc | Cy5 | 63 |
| | A3P2 | gcctg | | |
| | pncA-RC- | cgccgaggtcacccgggtcgatgtggaagtccttggtt | FAM | 64 |
| | A3P3 | gtcggcg | | |
| Amp4 | A4RCP1 | ctgcgcgcacccttgtagaacaccgcctcgattgccga | TxR | 65 |
| | | gcgcag | | |
| | A4RCP2 | ccgctcgattgccgacgtgtccagactgggatggagc | Cy5 | 66 |
| | | gg | | |
| | A4RCP3 | cgccgagtccgcgccgggagtaccgctgacgcaatc | FAM | 67 |
| | | ggcg | | |
| Amp5 | A5RCP1 | cgcgcattcagcagtggcgtgccgttctcgtcgactgc | TxR | 68 |
| | | gcg | | |
| | A5RCP2b | cgcgcgtcgactccttagaagccgctgtacgctccgcg | FAM | 69 |
| | | cg | | |
| Amp6 | A6RCP1 | cctgcacggcaacgcggcgtagatgaggtcgatgtgt | TxR | 70 |
| | | gcagg | | |
| | A6RCP2a | cgcggatcgatgtggtaggtattgcaaccgatcatttcc | Cy5 | 71 |
| | | gcg | | |
| Amp7 | A7RCP1 | cgtggattgcgtaccgcgtcctcggccgtctggcgcac | Cy5 | 72 |
| | | ccacg | | |
| | A7RCP2a | cccagacacaatgatcggtggcaataccgaccacatct | FAM | 73 |
| | | ggg | | |
| Amp8 | A8RCP1 | ctcgat(cy5)gctggcggtgcgcatctcctccagcg | Cy5 on | 74 |
| | | | internal T | |
| | A8RCP2 | cgcgctgcgacggtagtatcggccgacacaccagctg | TxR and FAM | 75 |
| | | agcgcg | | |
| | A8RCP3 | ccgtgccgacacacccgctgtcaggtccaccagcacg | TxR | 76 |
| | | cacgg | | |
| Amp1 | pncA1-RSA- | cggcgcgtcgatgatgatcaacgcccgcatacgtccac | TxR | 77 |
| | P3A | cgcgccg | | |
| Amp3 | pncA-RC- | cgcgctgacgcaatgcggtggccacgacgaagaata | TxR | 78 |
| | A3P1-65SM | gtagcgcg | | |
| Amp3 | pncARC- | acggcgaatagtccgttgtgccggagaagtggtcacc | Cy5 | 79 |
| | A3P2-mut | gccgt | | |
| Amp5 | A5RCP2 | cgcgcgtcgactccttcgaagccgctgtacgctccgcg | FAM | 80 |
| | | cg | | |
| Amp6 | A6RCP2b | cgcgagtcgatgtggtaggtattgcgaccgatcattctc | Cy5 | 81 |
| | | gcg | | |
| Amp7 | A7RCP2b | ccgcgacacaatgatcgatggcaataccgaccacatc | FAM | 82 |
| | | gcgg | | |

| Name | Sequence | SEQ ID NO |
|---|---|---|
| Amp-3 SM65-MB1 | ccgcaactattcttcgtcgtggtgcgg | 83 |
| Amp-3 WT65-MB1 | ccgagcacaccggactattcctcgtcgtggctcgg | 84 |
| pncA3-C65-slmt-RC-MB-1 | ccgagccacgacgaagaatagtccggtgtgctcgg | 85 |
| Amp-3 SM65-MB2 | ccgagactattcttcgtcgtggctcgg | 86 |
| Amp-3 SM65-MB3 | ccagcgactattcttcgtcgtggcgctgg | 87 |
| Amp-3 WT65-MB2 | ccgcaccggactattcctcgtcgtgcgg | 88 |
| pncA3-C65-slmt-RC-MB-2 | ccgcacgacgaagaatagtccggtgcgg | 89 |

What is claimed is:

1. A method for identifying one or more pyrazinamide-resistant mutations in the pncA gene of Mycobacterium tuberculosis, comprising:
amplifying a nucleic acid of Mycobacterium tuberculosis in a sample with one or more primer pairs to obtain one or more amplicons, wherein each of the one or more primer pairs comprises a forward primer and a reverse primer, wherein the one or more primer pairs is each specific for a target region of the nucleic acid, and wherein the one or more amplicons respectively correspond to one or more target regions of the nucleic acid;
contacting the one or more amplicons with one or more probes under a condition conducive to a hybridization reaction to form one or more probe-amplicon hybrids;
determining a melting temperature (Tm) of each of the one or more probe-amplicon hybrids;
determining a difference between the melting temperature of each of the one or more probe-amplicon hybrids and a reference melting temperature corresponding to the same probe-amplicon hybrid; and
identifying one or more pyrazinamide-resistant mutations in the nucleic acid in the sample based on the difference between the melting temperature of each of the one or more probe-amplicon hybrids and the reference melting temperature corresponding to the same probe-amplicon hybrid.

2. The method of claim 1, wherein the one or more primer pairs are adapted to amplify an antisense strand of the nucleic acid.

3. The method of claim 1, wherein the one or more probes hybridize to the antisense strand of the one or more amplicons.

4. The method of claim 1, wherein a mismatch caused by the one or more mutations within the one or more probe-amplicon hybrids is located at the center of the one or more probes.

5. The method of claim 1, wherein the difference is equal to or greater than 1 degree Celsius.

6. The method of claim 1, wherein the one or more primer pairs comprise six primer pairs.

7. The method of claim 1, wherein the one or more probes comprise 1 to 10 probes for each target region.

8. The method of claim 1, wherein one of more probes are designed to detect silent mutations and polymorphic mutations not conferring pyrazinamide resistance.

9. The method of claim 1, wherein the step of amplifying is performed by a polymerase chain reaction (PCR).

10. The method of claim 1, wherein the sample comprises a genomic DNA or fragment thereof of Mycobacterium tuberculosis.

11. The method of claim 10, comprising extracting the genomic DNA or fragment thereof from the sample.

12. The method of claim 1, wherein the step of amplifying for each of the one or more primer pairs is performed in separate reaction mixtures.

13. The method of claim 1, wherein at least one of the one or more target regions comprises one or more mutations that confer pyrazinamide resistance of Mycobacterium tuberculosis.

14. The method of claim 1, wherein at least one of the one of more target regions comprises one or more mutations that are silent mutations or polymorphic mutations that do not confer pyrazinamide resistance in Mycobacterium tuberculosis.

15. The method of claim 1, wherein the one or more primer pairs comprise a primer having a nucleotide sequence having at least 90% sequence identity with a nucleotide sequence of SEQ ID NOs: 1-25, or having a nucleotide sequence of SEQ ID NOs: 1-25.

16. The method of claim 1, wherein the one or more primer pairs comprise a forward and reverse primer sequence pair set forth respectively in SEQ ID NOs: 1-2; 1-3; 4-5; 6-7; 6-8; 9-10; 11-12; 11-13; 14-15; 16-17; 16-18; 19-20; 21-22; 23-24; and 23-25.

17. The method of claim 1, wherein the one or more probes comprise a probe having a nucleotide sequence having at least 90% sequence identity with a nucleotide sequence of SEQ ID NOs: 26-89, or having a nucleotide sequence of SEQ ID NOs: 26-89.

18. The method of claim 1, wherein the one or more probes include one or more labels located internally or at a terminus of the one or more probes.

19. The method of claim 18, wherein the one or more labels comprise at least one of a fluorophore and a quencher.

20. (canceled)

21. The method of claim 19, wherein the fluorophore is selected from fluorescein, cyanine 3, cyanine 5, TexasRed, and TAMRA, and wherein the quencher is selected from BHQ1, BHQ2, and DABCYL.

22-32. (canceled)
